(19) United States
(12) Patent Application Publication
(10) Pub. No.: US 2025/0019736 A1
(43) Pub. Date: Jan. 16, 2025

(54) MICROBIAL ELECTROLYSIS CELL (MEC) REACTOR AND RAPID TEST METHOD FOR BIOCHEMICAL OXYGEN DEMAND (BOD) OF ORGANIC WASTEWATER

(71) Applicant: Nankai University, Tianjin (CN)

(72) Inventors: Xin WANG, Tianjin (CN); Chengmei LIAO, Tianjin (CN); Ziyuan WANG, Tianjin (CN); Nan LI, Tianjin (CN)

(73) Assignee: Nankai University, Tianjin (CN)

(21) Appl. No.: 18/535,064

(22) Filed: Dec. 11, 2023

(30) Foreign Application Priority Data
Jul. 13, 2023 (CN) 2023108555055

(51) Int. Cl. C12Q 1/02; C12M 1/12; C12M 1/34; C12M 1/42

(52) U.S. Cl. CPC C12Q 1/02; C12M 25/14; C12M 35/02; C12M 41/46

(57) ABSTRACT

The present disclosure provides a microbial electrolysis cell (MEC) reactor and a rapid test method for a biochemical oxygen demand (BOD) of organic wastewater, and relates to the technical field of analysis and detection. In the present disclosure, an efficient MEC reactor is constructed based on a MEC sensing technology; meanwhile, combined with a continuous short-cycle test method and a supporting data calculation method, a rapid test method is obtained, and can be applied to various types of complex organic wastewater that does not have a biologically toxic substance and is greater than 5 mg BOD/L. After three cycles of test, last two stable cycles are taken to allow calculation; at the same time, a resulting curve is divided into multiple segments according to changes in main components of a degraded organic matter in different periods to allow segmented weighted calculations.

### CROSS-REFERENCE(S) TO RELATED APPLICATION(S)

This patent application claims the benefit and priority of Chinese Patent Application No. 2023108555055, filed with the China National Intellectual Property Administration on Jul. 13, 2023, the disclosure of which is incorporated by reference herein in its entirety as part of the present application.

### TECHNICAL FIELD

The present disclosure relates to the technical field of analysis and detection, and in particular to a microbial electrolysis cell (MEC) reactor and a rapid test method for a biochemical oxygen demand (BOD) of organic wastewater.

### BACKGROUND

Biochemical oxygen demand (BOD) refers to a level of organic matters in water that can be utilized by microorganisms, and plays an important role in sewage plant operation and maintenance as well as environmental monitoring.

A dilution and seeding method takes five days to test a biochemical oxygen demand after 5 days (BOD₅), and requires dilution, inoculation and other processing of a water sample to be tested. This method is also easily affected by temperature, water sample properties, and inoculation effect during the testing, and is not conducive to engineering applications. The differential pressure gauge-based BOD test technology that assists a differential pressure gauge also shows the same shortcomings.

Other existing indirect BOD test methods, such as microbial electrode method, microbial fuel cell sensing method and the like have long testing time and cannot guarantee stability. Especially when testing the BOD of organic wastewater containing complex material components, there is a relatively big error between the test results and a national standard BOD₅ value, indicating poor reproducibility of the data.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

An objective of the present disclosure is to provide a microbial electrolysis cell (MEC) reactor and a rapid test method for a biochemical oxygen demand (BOD) of organic wastewater. The present disclosure is suitable for rapid detection of various types of complex organic wastewater that does not have a biologically toxic substance, and has desirable data reproducibility and a small error in test results compared with those of the national standard BOD₅ value.

To achieve the above objective, the present disclosure provides the following technical solutions:

The present disclosure provides a MEC reactor, including a reactor body, a bioanode, and a cathode; where the bioanode is located at a center in a cavity of the reactor body; the cathode wraps but does not contact the bioanode, and a hollow cavity between the bioanode and the cathode forms an electrolysis chamber; and the reactor body is provided with a water inlet and a water outlet; and
the bioanode includes a bioaffinity material and a functional microorganism attached to the bioaffinity material; the functional microorganism includes an electroactive microorganism and a mixed culture; the electroactive microorganism is purified Geobacter sulfurreducens; and the mixed culture at least simultaneously includes Methanobacterium, Methanocorpusculum, Propionicimonas, and Klebsiella.

Preferably, a volume of the bioanode accounts for not less than 60% of that of the cavity of the reactor body.

Preferably, the cathode is in close contact with an inner wall of the reactor body.

Preferably, the cathode is prepared from a titanium-based material; and the titanium-based material has titanium with a mass content of greater than 80%.

Preferably, the cathode is in a mesh structure, and the mesh structure has a mesh number of not less than 100 mesh.

Preferably, the bioaffinity material is a carbon-based material.

The present disclosure further provides a rapid test method for a BOD of organic wastewater, including the following steps:
filling the MEC reactor with a nutrient solution that contains sodium acetate and does not contain dissolved oxygen, replacing the nutrient solution in a cycle of every 1 d to 3 d, detecting a current change in each cycle, and deeming that the MEC reactor has been started successfully when a difference between current peaks of adjacent cycles is less than 5%; and
repeating the following operations three times: pouring organic wastewater to be tested into the electrolysis chamber of the MEC reactor that has been started successfully through the water inlet, and then discharging the organic wastewater to be tested through the water outlet after staying for 10 min to 40 min; recording current data generated during each time of the operations; averaging resulting time-current curves obtained from last two times to obtain an averaged time-current curve; fitting the averaged time-current curve with an exponential decay curve to obtain a fitted time-current curve with a termination current of 0.01 mA to 0.1 mA; and dividing the fitted time-current curve into n+1 characteristic declining stages according to a number n of inflection points in the fitted time-current curve, and calculating the BOD with reference to Formula 1;

$$BOD = \frac{{Q}_{0} + {\sum }_{i = 1}^{n + 1} ( n + 1 ) {ε}_{i} {\int }_{{t}_{i - 1}}^{{t}_{i}} {I}_{F} d t}{F σ} ; \qquad Formula 1$$

where
in Formula 1: Q₀ represents a reference electric quantity of the MEC reactor, in C; when the volume of the bioanode accounts for not less than 80% of that of the cavity of the reactor body, Q₀ is 3,000 C; when the volume of the bioanode accounts for 60% to 80% of that of the cavity of the reactor body, Q₀=150V+4200, and V represents a volume ratio, in %;
n represents the number of inflection points in the fitted time-current curve with a value of 0 to 2 according to an actual situation, and is dimensionless;
εᵢ represents a weighting coefficient for each of the characteristic declining stages in the fitted time-current curve, and is dimensionless; when n=0, i=1, ε₁=1; when n=1, i=1, 2, ε₁=0.4, ε2=0.6; and when n=2, i=1, 2, 3, ε₁=0.2, ε2=0.2, 83=0.6;
F represents a Faraday's constant, 96,500 C/mol;
σ represents a total oxygen efficiency, in mol ·L/mg;
I_{F} represents a fitted curve, in mA;
tᵢ₋₁ represents a starting time point of an i-th characteristic decline curve, in s;
tᵢ represents an ending time point of the i-th characteristic decline curve, in s;
BOD represents a calculated BOD value, in mg/L; and
a calculation process of the total oxygen efficiency σ includes: determining a BOD₅ value of the organic wastewater to be tested using a dilution and seeding method, and then substituting the BOD₅ value into Formula 1 to calculate the total oxygen efficiency σ.

Preferably, before the organic wastewater to be tested is introduced, the method further includes: draining a liquid in the MEC reactor, and cleaning the cavity of the MEC reactor with deionized water.

Preferably, the nutrient solution has a pH value of 7.2; and the nutrient solution has the sodium acetate at a concentration of 0.1 mg/L to 0.5 mg/L.

Preferably, the organic wastewater to be tested has a BOD₅ value of greater than 5 mg/L and does not have a biologically toxic substance.

The present disclosure provides a MEC reactor, including a reactor body, a bioanode, and a cathode; where the bioanode is located at a center in a cavity of the reactor body; the cathode wraps but does not contact the bioanode, and a hollow cavity between the bioanode and the cathode forms an electrolysis chamber; and the reactor body is provided with a water inlet and a water outlet; and the bioanode includes a bioaffinity material and a functional microorganism attached to the bioaffinity material; the functional microorganism includes an electroactive microorganism and a mixed culture; the electroactive microorganism is purified Geobacter sulfurreducens; and the mixed culture at least simultaneously includes Methanobacterium, Methanocorpusculum, Propionicimonas, and Klebsiella.

During the MEC test for BOD, the electroactive microorganism can consume BOD and generate electrons; the electrons are transferred to the electrodes to generate an electrical signal, and BOD information can be obtained by analyzing the electrical signal. In the present disclosure, the purified Geobacter sulfurreducens is used as one of the functional microorganisms attached to the bioanode, thereby ensuring that the electroactive microorganism in the functional microorganism have a high relative abundance. In addition, a mixed culture is further added to the bioanode, and has the functions of producing methane, producing acetic acid, and degrading propionic acid. The mixed culture is rich in species, can adapt to a variety of water characteristics, and can convert complex organic matters into acetic acid and other substrates that are easily utilized by the electroactive microorganism. In this way, a functional microbial membrane of the bioanode can consume more complex organic matters and generate stronger electrical signals. Therefore, the MEC reactor shows a wide detection range and can test different organic wastewater containing complex substances.

In the present disclosure, an efficient MEC reactor is constructed based on a MEC sensing technology; meanwhile, combined with a continuous short-cycle test method and a supporting data calculation method, a rapid test method is obtained, and can be applied to various types of complex organic wastewater that does not have a biologically toxic substance and is greater than 5 mg BOD/L. After three cycles of test, last two stable cycles are taken to allow calculation; at the same time, a resulting curve is divided into multiple segments according to changes in main components of a degraded organic matter in different periods to allow segmented weighted calculations. Compared with an ordinary coulomb method, this calculation method can more accurately combine a coulomb electric charge with the BOD of a dilution and seeding method from the perspective of an actual microbial electrochemical metabolism. This method has desirable data reproducibility, and a test result error with a national standard BOD₅ value of less than 10%.

In addition, in the present disclosure, when testing the BOD of organic wastewater, only suspended solids of large particle size need to be filtered out, without the need for other pre-treatments such as dilution, showing simple operations.

### DESCRIPTION OF THE DRAWINGS

FIG. 1 shows a schematic structural diagram of the MEC reactor; reference numerical: 1-water outlet, 2-bioanode, 3-cathode, 4-reactor body, 5-electrode lead open, 6-water inlet;

FIG. 2 shows last two time-current error band curves of two water samples to be tested in Example 1;

FIG. 3 shows last two time-current error band curves in Example 2; and

FIG. 4 shows a three-cycle time-current curve of a glucose-glutamic acid standard sample test in Example 3.

The foregoing aspects and many of the attendant advantages of this invention will become more readily appreciated as the same become better understood by reference to the following detailed description, when taken in conjunction with the accompanying drawings, wherein:

### DETAILED DESCRIPTION

The present disclosure provides a MEC reactor, including a reactor body, a bioanode, and a cathode; where the bioanode is located at a center in a cavity of the reactor body; the cathode wraps but does not contact the bioanode, and a hollow cavity between the bioanode and the cathode forms an electrolysis chamber; and the reactor body is provided with a water inlet and a water outlet; and

the bioanode includes a bioaffinity material and a functional microorganism attached to the bioaffinity material; the functional microorganism includes an electroactive microorganism and a mixed culture; the electroactive microorganism is purified Geobacter sulfurreducens; and the mixed culture at least simultaneously includes Methanobacterium, Methanocorpusculum, Propionicimonas, and Klebsiella.

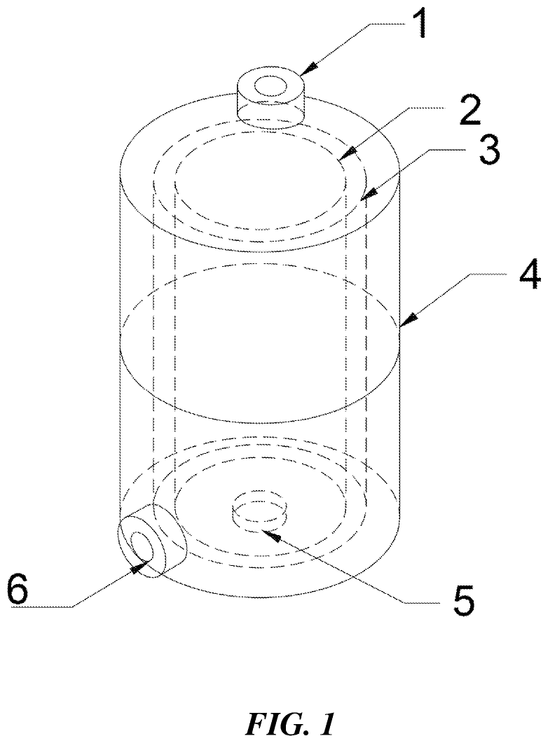

In the present disclosure, a structure of the MEC reactor will be described below with reference to FIG. 1.

As shown in FIG. 1, the MEC reactor provided by the present disclosure includes a reactor body. In the present disclosure, the reactor body is preferably a hollow cylinder; the reactor body is provided with a water inlet and a water outlet; more specifically, the water inlet is provided on a lower side of the reactor body, and the water outlet is provided on a top surface of the reactor body. The reactor body has an inner diameter of preferably 20 mm to 40 mm, more preferably 25 mm to 30 mm; and the reactor body has a height of preferably 40 mm to 60 mm, more preferably 50 mm. There are no special requirements for a material of the reactor body; in an example, the reactor body is prepared from an engineering plastic.

In the present disclosure, the MEC reactor includes a bioanode; the bioanode is located at a center in a cavity of the reactor body, that is, a vertical central axis of the bioanode coincides with a central axis of the reactor body. The bioanode is preferably cylindrical, but may also be in other shapes, such as rectangular parallelepiped, and can be freely adjusted by those skilled in the art. A height of the bioanode is preferably consistent with that of the reactor body; a volume of the bioanode accounts for preferably not less than 60%, more preferably not less than 80%, and even more preferably 80% to 90% of that of the cavity of the reactor body. A volume ratio of the bioanode can be adjusted by adjusting its diameter. By controlling the volume ratio of the bioanode to not less than 80%, a higher inoculation amount of functional microorganism is ensured, which can further improve the speed and accuracy of BOD detection. When the volume ratio is less than 80%, an error between the test results and a national standard dilution and seeding method results may increase significantly; when the volume ratio is less than 60%, it is considered that the bioreactor is not suitable for BOD rapid test in the present disclosure.

In the present disclosure, the bioanode includes a bioaffinity material and a functional microorganism attached to the bioaffinity material; the functional microorganism includes an electroactive microorganism and a mixed culture; the electroactive microorganism is purified Geobacter sulfurreducens; and the mixed culture at least simultaneously includes Methanobacterium, Methanocorpusculum, Propionicimonas, and Klebsiella.

In the present disclosure, on one hand, the purified Geobacter sulfurreducens is used as the electroactive microorganism, thereby ensuring that the electroactive microorganism in the functional microorganism has a high relative abundance, which is conducive to the generation of strong electrical signals. On the other hand, a mixed culture is further added to the bioanode, and has the functions of producing methane, producing acetic acid, and degrading propionic acid. The mixed culture is rich in species, can adapt to a variety of water characteristics, and can convert complex organic matters into acetic acid and other substrates that are easily utilized by the electroactive microorganism. In this way, a functional microorganism membrane of the bioanode can consume more complex organic matters and generate stronger electrical signals. Therefore, the MEC reactor shows a wide detection range and can test the BOD values of different organic wastewater containing complex substances.

In the present disclosure, the bioaffinity material is preferably a carbon-based material; the carbon-based material preferably includes a cylinder, a porous graphite column, or a stone grinding rod that is stacked with carbon fiber filaments, more preferably the porous graphite column. The bioaffinity material is configured to attach the functional microorganism. The bioaffinity material is preferably ultrasonically cleaned with acetone, ethanol, and ultrapure water before use, and then dried at a room temperature.

In the present disclosure, there are no special requirements for a preparation process of the bioanode, and the microbial inoculation method well known in the art is used for inoculation and preparation; in an example, the preparation process of the bioanode includes preferably the following steps:
1, pure bacteria inoculation: resuscitating and activating the purified Geobacter sulfurreducens stored in a −80° C. refrigerator in a nutrient broth medium for 12 h, placing 100 mL of a liquid bacterial solution into a 30° C. incubator to allow activation under sterile conditions, and then inoculating into the MEC reactor at OD₆₀₀=0.1;
2, after the pure bacteria inoculation is completed, conducting culture until a current naturally rises and then drops to less than 0.1 mA;
3, pure bacteria culture: replacing a nutrient solution with pH=7.2, containing sodium acetate 0.3 mg/L, and showing DO<0.1 mg/L into the MEC reactor, replacing with a new nutrient solution when the current in the MEC reactor is detected to be less than 0.1 mA, and repeating the above operations twice;
4, mixed culture inoculation: adding 0.8 mg/L sodium acetate into a sludge leachate of a cultured mixed culture, adjusting to a pH value of 7.2 and DO<0.1 mg/L to obtain a functional bacteria inoculum solution, replacing the functional bacteria inoculum solution into the MEC reactor, and replacing with a new inoculation solution when the current in the MEC reactor is detected to be less than 0.1 mA; and
5, culture: replacing the nutrient solution with pH=7.2, containing sodium acetate 0.3 mg/L, and showing DO<0.1 mg/L into the MEC reactor, replacing with a new nutrient solution when the current in the MEC reactor is detected to be less than 0.1 mA, and repeating the above operations until a difference between current peaks is less than 5%, such that the bioanode is successfully prepared.

In the present disclosure, there are no special requirements for a source of the purified Geobacter sulfurreducens, which can be obtained by using purification methods well known in the art; specifically, this strain is isolated and purified from a biofilm of the MEC bioanode cultured in a laboratory for 2 years. The mixed culture is domesticated from domestic sewage and fermentation wastewater in the laboratory. Preferably, 16s rDNA sequencing is conducted on the bioanode membrane, and four genera of Methanobacterium, Methanocorpusculum, Propionicimonas, and Klebsiella are detected.

In the present disclosure, the MEC reactor includes a cathode; the cathode wraps but does not contact the bioanode, and a hollow cavity between the bioanode and the cathode forms an electrolysis chamber. The cathode is preferably in close contact with an inner wall of the reactor body. The cathode is prepared from preferably a titanium-based material; and the titanium-based material has titanium with a mass content of preferably greater than 80%. The titanium-based material is the cathode and has excellent electrical conductivity and catalytic performance. The cathode is preferably in a mesh structure, and the mesh structure has a mesh number of preferably not less than 100 mesh. The mesh structure facilitates hydraulic shearing while taking away electrode reaction products near the metal mesh, promoting the reaction to proceed in the positive direction; at the same time, the mesh structure is less likely to cause clogging and can save costs. A height of the cathode is preferably consistent with that of the reactor body.

As an example of the present disclosure, an electrode lead open is provided on a bottom surface of the MEC reactor, the electrode lead open is configured to lead out leads of the bioanode and the cathode, and the leads are connected to a power supply.

In the present disclosure, during actual test, the MEC reactor, a microcurrent acquisition card, and a sampling pump are assembled into a complete BOD sensor for later use. More specifically, positive and negative poles of a DC power supply of 0.5 V to 1.0 V are connected to the bioanode and the cathode of the MEC reactor, respectively, and the microcurrent acquisition card is connected in series in a circuit to collect electrical signals; a water inlet of the sampling pump is connected to a water sample to be tested, while a water outlet of the sampling pump is connected to the water inlet of the MEC reactor.

The present disclosure further provides a rapid test method for a BOD of organic wastewater, including the following steps:
filling the MEC reactor with a nutrient solution that contains sodium acetate and does not contain dissolved oxygen, replacing the nutrient solution in a cycle of every 1 d to 3 d, detecting a current change in each cycle, and deeming that the MEC reactor has been started successfully when a difference between current peaks of adjacent cycles is less than 5%; and
repeating the following operations three times: pouring organic wastewater to be tested into the electrolysis chamber of the MEC reactor that has been started successfully through the water inlet, and then discharging the organic wastewater to be tested through the water outlet after staying for 10 min to 30 min; recording current data generated during each time of the operations; averaging resulting time-current curves obtained from last two times to obtain an averaged time-current curve; fitting the averaged time-current curve with an exponential decay curve to obtain a fitted time-current curve having a termination current of 0.01 mA to 0.1 mA; and dividing the fitted time-current curve into n+1 characteristic declining stages according to a number n of inflection points in the fitted time-current curve, and calculating the BOD with reference to Formula 1;

$$BOD = \frac{{Q}_{0} + {\sum }_{i = 1}^{n + 1} ( n + 1 ) {ε}_{i} {\int }_{{t}_{i - 1}}^{{t}_{i}} {I}_{F} d t}{F σ} ; \qquad Formula 1$$

where
in Formula 1: Q₀ represents a reference electric quantity of the MEC reactor, in C; when the volume of the bioanode accounts for not less than 80% of that of the cavity of the reactor body, Q₀ is 3,000 C; when the volume of the bioanode accounts for 60% to 80% of that of the cavity of the reactor body, Q₀=150V+4200, and V represents a volume ratio, in %;
n represents the number of inflection points in the fitted time-current curve with a value of 0 to 2 according to an actual situation, and is dimensionless;
εᵢ represents a weighting coefficient for each of the characteristic declining stages in the fitted time-current curve, and is dimensionless; when n=0, i=1, ε₁=1; when n=1, i=1, 2, ε₁=0.4, ε₂=0.6; and when n=2, i=1, 2, 3, ε₁=0.2, ε2=0.2, ε₃=0.6;
F represents a Faraday's constant, 96,500 C/mol;
σ represents a total oxygen efficiency, in mol ·L/mg;
I_{F} represents a fitted curve, in mA;
tᵢ₋₁ represents a starting time point of an i-th characteristic decline curve, in s;
tᵢ represents an ending time point of the i-th characteristic decline curve, in s;
BOD represents a calculated BOD value, in mg/L; and

In the present disclosure, the MEC reactor is filled with a nutrient solution that contains sodium acetate and does not contain dissolved oxygen, the nutrient solution is replaced in a cycle of every 1 d to 3 d, preferably every 2 d, a current change in each cycle is detected, and it is deemed that the MEC reactor has been started successfully when a difference between current peaks of adjacent cycles is less than 5%.

In the present disclosure, the nutrient solution has a pH value of preferably 7.2; and the nutrient solution has the sodium acetate at a concentration of preferably 0.1 mg/L to 0.5 mg/L, more preferably 0.2 mg/L to 0.3 mg/L. The functional microorganism is strictly anaerobic or facultative anaerobic, and oxygen may interfere with the enrichment and colonization of functional bacteria in the early stages of culture. Therefore, the nutrient solution cannot contain dissolved oxygen, but there is no need to ensure that the water sample is strictly anaerobic during actual test after the culture is mature.

In the present disclosure, the current change in each cycle is preferably detected by the microcurrent acquisition card.

In the present disclosure, after the MEC reactor is successfully started, the liquid in the MEC reactor is preferably drained, the cavity of the MEC reactor is cleaned with deionized water, and then the organic wastewater is introduced for detection.

In the present disclosure, there are no special requirements for a deionized water cleaning process. The deionized water is directly filled into the electrolysis chamber of the MEC reactor and stayed for 5 min before being discharged.

In the present disclosure, the organic wastewater to be tested has a BOD₅ of preferably less than 5 mg/L and does not have a biologically toxic substance. There are no special requirements for a composition of the organic wastewater to be tested, and it is suitable for organic wastewater with a complex composition. When the organic wastewater to be tested is a water body containing suspended matters with a larger particle size, it is preferred to pass through a 20-mesh filter before injecting the sample. The method does not require any pre-treatment, is simpler to operate and has a shorter process than existing detection methods.

In the present disclosure, the following operations are repeated three times: organic wastewater to be tested is poured into the electrolysis chamber of the MEC reactor that has been started successfully through the water inlet, and then the organic wastewater to be tested is discharged through the water outlet after staying for 10 min to 40 min; current data generated during each time of the operations are recorded; resulting time-current curves obtained from last two times are averaged to obtain an averaged time-current curve.

In the present disclosure, an amount of the organic wastewater to be tested is preferably such that the electrolysis chamber is filled up. The last two time-current curves are stable and repeatable, and represent a real situation of water sample test. There is a relatively big current curve error in a first cycle, indicating a process of the bioanode adapting to the water sample. Therefore, averaging the time-current curves obtained from the last two tests to obtain the averaged time-current curve can ensure the accuracy and desirable reproducibility of the data.

In the present disclosure, the averaged time-current curve is fitted with an exponential decay curve to obtain a fitted time-current curve having a termination current of 0.01 mA to 0.1 mA; and the BOD is calculated with reference to Formula 1.

In the present disclosure, since the current continuously decays with time during actual detection, fitting the exponential decay curve is more consistent with the actual situation. There are no special requirements for a fitting process, and a fitting process well known in the art can be used. The fitted time-current curve has a termination current of preferably 0.01 mA to 0.1 mA, more preferably 0.01 mA to 0.05 mA.

In the present disclosure, the fitted time-current curve is divided into n+1 characteristic declining stages according to a number n of inflection points (zero points of the second derivative) in the fitted time-current curve, and the BOD is calculated with reference to Formula 1.

In the present disclosure, a calculation process of the total oxygen efficiency σ in Formula 1 includes: determining a BOD₅ value of the organic wastewater to be tested using a dilution and seeding method, and then substituting the BOD₅ value into Formula 1 to calculate the total oxygen efficiency σ.

In the present disclosure, it is preferable to determine the BOD₅ values of multiple organic wastewater samples, calculate multiple σ values, and then calculate an average σ value as a σ value during subsequent actual detection.

In the present disclosure, an accuracy of the detection data can be ensured by using the dilution and seeding method to calibrate the σ value.

In the present disclosure, based on a coulomb method, the time-current curves generated after the last two sample injections are averaged, and the BOD value is calculated through fitting prediction, piecewise integration, and weighted summation. Compared with an ordinary coulomb method, this calculation method can more accurately combine a coulomb electric charge with the BOD of a dilution and seeding method from the perspective of an actual microbial electrochemical metabolism. This method has desirable data reproducibility, and a test result error with a national standard BOD₅ value of less than 10%.

In the present disclosure, the above calculation method and formula are preferably built into the microcurrent acquisition card, and the microcurrent acquisition card automatically outputs the BOD value.

In the present disclosure, when the MEC reactor is not used for a long time, it is necessary to introduce a nutrient solution with pH=7.2 and containing 2 mg/L sodium acetate into the cavity of the MEC reactor to put the device in a maintenance state; this nutrient solution is replaced once a month. In this way, the MEC reactor can be used for testing without restarting the next time it is used.

The MEC reactor and a rapid test method for a BOD of organic wastewater provided by the present disclosure are described in detail below with reference to the examples, but these examples may not be understood as a limitation to the protection scope of the present disclosure.

In the following examples, a structure of the MEC reactor used is shown in FIG. 1. The reactor body is a hollow cylinder with an inner diameter of 30 mm and a height of 60 mm, and is prepared from an engineering plastic. The bioanode is located at a center in a cavity of the reactor body, and its height is consistent with that of the reactor body, and the bioanode includes a bioaffinity material, which is a porous graphite column. A functional microorganism is attached to the porous graphite column; the functional microorganism includes purified Geobacter sulfurreducens and a mixed culture; the mixed culture at least simultaneously includes Methanobacterium, Methanocorpusculum, Propionicimonas, and Klebsiella. A specific preparation method of the bioanode has been discussed previously and will not be repeated here; a volume of the bioanode is 90% of that of the cavity of the reactor body. The cathode is a mesh prepared from pure titanium and is close to an inner wall of the reactor body; a height of the cathode is consistent with that of the reactor body; the cathode has a mesh number of 100 mesh.

In the present disclosure, the MEC reactor, a microcurrent acquisition card, and a sampling pump are assembled into a complete BOD sensor for later use. More specifically, positive and negative poles of a DC power supply of 1.0 V are connected to the bioanode and the cathode of the MEC reactor, respectively, and the microcurrent acquisition card is connected to the bioanode and the cathode of the MEC reactor in series in a circuit to collect electrical signals; a water inlet of the sampling pump is connected to a water sample to be tested, while a water outlet of the sampling pump is connected to the water inlet of the MEC reactor.

### Example 1

Test of Fermentation Wastewater with BOD₅ Value of about 6,000 mg/L

### (1) Parameter Solution

A BOD sensor was prepared, sampling was conducted to collect time-current curves according to the aforementioned process; last two time-current curves were averaged to obtain an averaged time-current curve; the averaged time-current curve was fitted to obtain a fitted time-current curve; since there were 2 inflection points in the fitted time-current curve when testing the fermentation wastewater, the curve was divided into 3 stages according to time points corresponding to the 2 inflection points, and there were &1, 2-0.2, and 83=0.6. Within 1 h, a total of 3 groups of fermentation wastewater samples at different time points were taken and tested separately, and dilution and seeding method BOD₅ values of the 3 groups of fermentation wastewater samples were tested at the same time; the relevant data were substituted into Formula 1, the σ values were calculated separately, and then averaged to obtain σ=0.052, which was then substituted into Formula 1 for subsequent calculations.

### (2) Sample Test

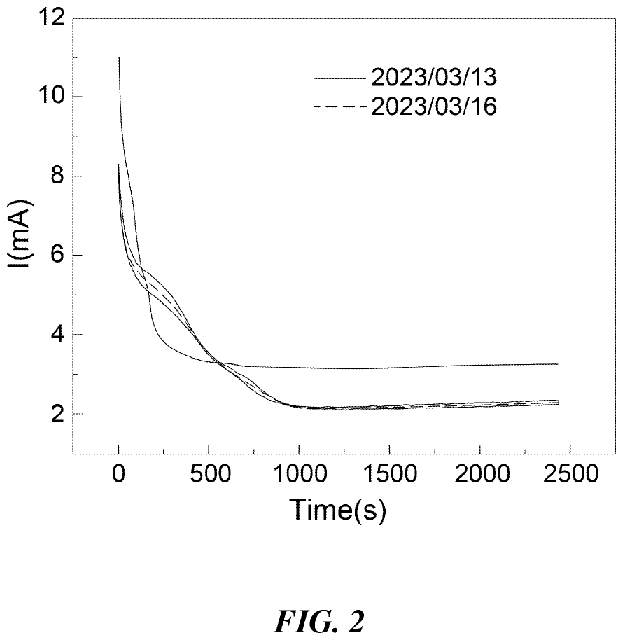

Samples of the fermentation wastewater were taken on Mar. 13, 2023 and Mar. 16, 2023, respectively, the following operations were repeated three times: fermentation wastewater to be tested was poured into the electrolysis chamber of the MEC reactor that had been started successfully through the water inlet, and then the fermentation wastewater to be tested was discharged through the water outlet after staying for 40 min; current data generated during each time of the operations were recorded using a microcurrent acquisition card; resulting time-current curves obtained from last two times were averaged to calculate a standard deviation, to obtain an averaged time-current curve; an error band diagram was made, as shown in FIG. 2; the averaged time-current curve was fitted with an exponential decay curve to obtain a fitted time-current curve; where the fitted curve for March 16 had the formula as follows:

$$\begin{matrix}I = {5.15}_{e}^{\frac{- t}{102}} + 1.48 \# ( 0 \leq t \leq 250 ) \\ I = 0.13 {e}^{\frac{- t}{- 393}} + 1.94 \# ( 250 \leq t \leq 450 ) \\ I = 2.33 {e}^{\frac{- t}{19918}} \# ( 450 < t \leq 108402 )\end{matrix}$$

The fitted time-current curve had a termination current of 0.1 mA; after substituting into the BOD calculation formula, the BOD values were obtained as 5,980 mg/L and 5,000 mg/L, and their errors with the national standard dilution inoculation method BOD₅ (5,720 mg/L, 4,890 mg/L) were 4.5% and 2.2%, respectively.

In addition, the results in FIG. 2 showed that: during the two water sample tests, the error between the time-current curves of the last two cycles was small, and an original curve of the test was stable; the test results for fermentation wastewater containing complex organic matters at different time periods were fully reliable, with a total test time of 40 min. These indicated that the test time of the present disclosure was short.

### Example 2

Test of deeply treated effluent with BOD₅ value of about 10 mg/L

### (1) Parameter Solution

A BOD sensor was prepared, according to the small BOD characteristics of the tested water sample, a single cycle was shortened to 10 min; sampling was conducted to collect time-current curves according to the aforementioned process; last two time-current curves were averaged to obtain an averaged time-current curve; the averaged time-current curve was fitted to obtain a fitted time-current curve; since there was 1 inflection point in the fitted time-current curve, the curve was divided into 2 stages according to time points corresponding to the 1 inflection points, and there were ε₁=0.4 and ε₂=0.6. Within 1 h, a total of 3 groups of fermentation wastewater samples at different time points were taken and tested separately, and dilution and seeding method BOD₅ values of the 3 groups of fermentation wastewater samples were tested at the same time; the relevant data were substituted into formula, the σ values were calculated separately, and then averaged to obtain σ=0.75, which was then substituted into the formula for subsequent calculations.

### (2) Sample Test

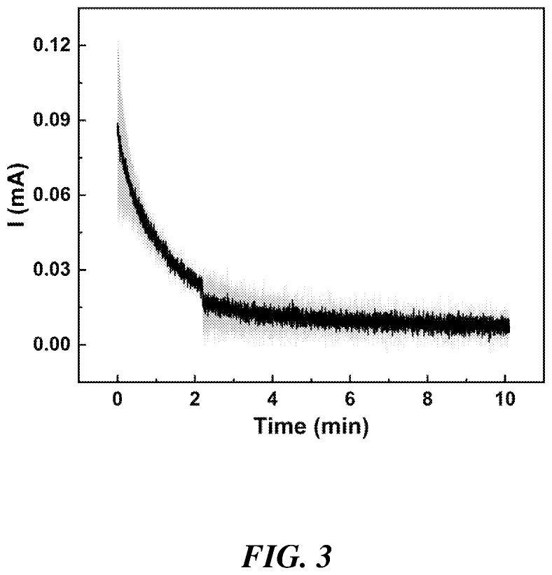

100 mL of water sample was taken from the deeply treated effluent, the following operations were repeated three times: the water sample was poured into the electrolysis chamber of the MEC reactor that had been started successfully through the water inlet, and then the fermentation wastewater to be tested was discharged through the water outlet after staying for 10 min; current data generated during each time of the operations were recorded using a microcurrent acquisition card; resulting time-current curves obtained from last two times were averaged to calculate a standard deviation, to obtain an averaged time-current curve; an error band diagram was made, as shown in FIG. 3; the averaged time-current curve was fitted with an exponential decay curve to obtain a fitted time-current curve; where the fitted time-current curve had the formula as follows:

$$\begin{matrix}I = {0.066}_{e}^{\frac{- t}{3980}} + 0.015 \# ( 0 \leq t \leq 150 ) \\ \begin{matrix}I = 0.019 {e}^{\frac{- t}{10796}} & ( 150 < t \leq 600 )\end{matrix}\end{matrix}$$

After substituting into the BOD calculation formula, the test result was 12 mg/L. Compared with the national standard dilution inoculation method BOD₅=13 mg/L, the test result error of this method was 7.7%, a total test time was 30 min, and the test was efficient and reliable.

### Example 3

Laboratory test of glucose-glutamic acid standard sample with BOD₅=250 mg/L

(1) Parameter solution

A BOD sensor was prepared as described in the method, a program of automatic periodic sampling and data processing was started, the glucose-glutamic acid standard sample with BOD₅=250 mg/L was introduced into the electrolysis chamber; since there were 0 inflection points of the fitted time-current curve, then the curve was divided into 1 stage according to the time point corresponding to the 0 inflection points, and there was ε₁=1. Within 1 h, a total of 3 groups of fermentation wastewater samples at different time points were taken and tested separately, and dilution and seeding method BOD₅ values of the 3 groups of fermentation wastewater samples were tested at the same time; the relevant data were substituted into formula, the σ values were calculated separately, and then averaged to obtain σ=0.8, which was then substituted into the formula for subsequent calculations.

### (2) Sample Test

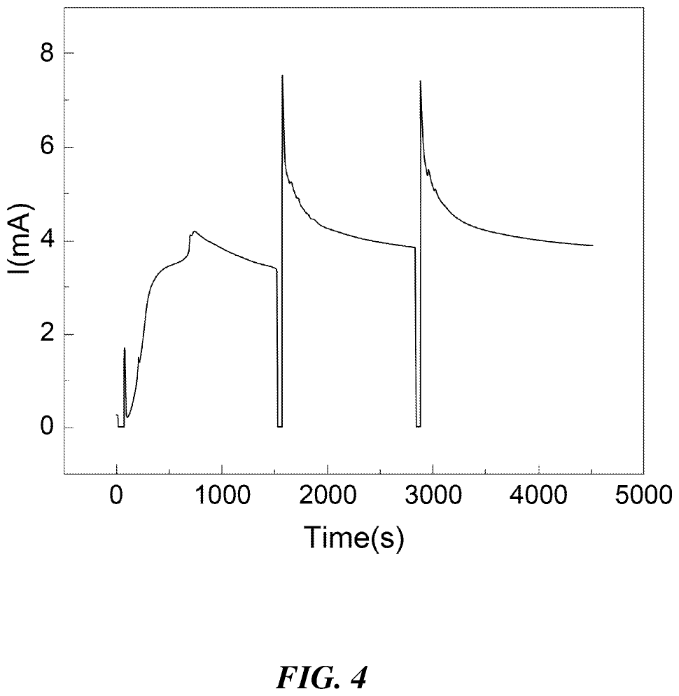

100 mL of the glucose-glutamic acid standard sample with BOD₅=250 mg/L was taken, and then injected for test according to the aforementioned method, where time-current curves of three cycles were shown in FIG. 4; last two time-current curves were averaged to obtain an averaged time-current curve; the averaged time-current curve was fitted with an exponential decay curve to obtain a fitted time-current curve; after substituting into the BOD calculation formula, a test result was 240 mg/L, with an error of 4%.

It was seen from the results of the above examples that the test results of different water samples using the method of the present disclosure were stable and reliable, and no pre-treatment of the water samples was required.

### Example 4

Comparison with differential pressure gauge-based BOD tester

For fermentation wastewater with BOD₅>5,000 mg/L, when tested using conventional differential pressure gauge-based BOD testers on the market, it was necessary to go through steps such as dilution and inoculation, adding nitrification inhibitors, and adding carbon dioxide absorbers. The wastewater had a complex organic composition, and it was difficult to accurately determine the range of BOD₅ through chemical oxygen demand (COD). Therefore, multiple dilution concentrations were generally required for testing, and each testing process took five days; moreover, the test results were easily affected by the ambient temperature and the tightness of the pressure differential test system.

In contrast, the solution of the present disclosure adopted mature biological electrodes as sensing elements, and did not require complex pre-treatment operations such as dilution and inoculation of water samples for organic wastewater of different compositions. The entire test process only required starting a sampling program, updating the water sample into the electrolytic cell at a cycle of 10 min to 20 min, and then reading the data at the end.

### Example 5

Comparison with microbial electrode method-based rapid detector

For water samples from biochemical pools in sewage plants with BOD₅<5,000 mg/L, BOD₅ test was conducted using a microbial electrode method-based BOD rapid tester with immobilized Escherichia coli and the like. An error between the results obtained and the BOD₅ results obtained by the national standard dilution inoculation method was easily greater than 10%; especially when the instrument was used for more than 1 month, the error might continue to increase.

In contrast, the solution of the present disclosure adopted functional microorganism communities for testing, and could maintain the stability of the sensing element for a longer period of time. A single component could maintain a repeatability deviation of less than 10% for 3 to 6 months (for example, in the present disclosure, the BOD value of the 0.5 g/L sodium acetate sample tested on Dec. 11, 2022 was 260 mg/L, while the BOD value of the 0.5 g/L sodium acetate sample tested on Jun. 1, 2023 was 250 mg/L, and there was no significant difference between the two). At the same time, the segmented integral calculation method could ensure that the error between the test results and the national standard dilution inoculation method BOD₅ was less than 10%.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

While illustrative embodiments have been illustrated and described, it will be appreciated that various changes can be made therein without departing from the spirit and scope of the invention.

1. A microbial electrolysis cell (MEC) reactor, comprising a reactor body, a bioanode, and a cathode; wherein the bioanode is located at a center in a cavity of the reactor body; the cathode wraps but does not contact the bioanode, and a hollow cavity between the bioanode and the cathode forms an electrolysis chamber; and the reactor body is provided with a water inlet and a water outlet; and
the bioanode comprises a bioaffinity material and a functional microorganism attached to the bioaffinity material; the functional microorganism comprises an electroactive microorganism and a mixed culture; the electroactive microorganism is purified Geobacter sulfurreducens; and the mixed culture at least simultaneously comprises Methanobacterium, Methanocorpusculum, Propionicimonas, and Klebsiella.

2. The MEC reactor according to claim 1, wherein a volume of the bioanode accounts for not less than 60% of that of the cavity of the reactor body.

3. The MEC reactor according to claim 1, wherein the cathode is in close contact with an inner wall of the reactor body.

4. The MEC reactor according to claim 2, wherein the cathode is in close contact with an inner wall of the reactor body.

5. The MEC reactor according to claim 1, wherein the cathode is prepared from a titanium-based material; and the titanium-based material has titanium with a mass content of greater than 80%.

6. The MEC reactor according to claim 1, wherein the cathode is in a mesh structure, and the mesh structure has a mesh number of not less than 100 mesh.

7. The MEC reactor according to claim 5, wherein the cathode is in a mesh structure, and the mesh structure has a mesh number of not less than 100 mesh.

8. The MEC reactor according to claim 1, wherein the bioaffinity material is a carbon-based material.

9. A rapid test method for a biochemical oxygen demand (BOD) of organic wastewater, comprising the following steps:
filling the MEC reactor according to claim 1 with a nutrient solution that contains sodium acetate and does not contain dissolved oxygen, replacing the nutrient solution in a cycle of every 1 d to 3 d, detecting a current change in each cycle, and deeming that the MEC reactor has been started successfully when a difference between current peaks of adjacent cycles is less than 5%; and
repeating the following operations three times: pouring organic wastewater to be tested into the electrolysis chamber of the MEC reactor that has been started successfully through the water inlet, and then discharging the organic wastewater to be tested through the water outlet after staying for 10 min to 40 min; recording current data generated during each time of the operations; averaging resulting time-current curves obtained from last two times to obtain an averaged time-current curve; fitting the averaged time-current curve with an exponential decay curve to obtain a fitted time-current curve with a termination current of 0.01 mA to 0.1 mA; and dividing the fitted time-current curve into n+1 characteristic declining stages according to a number n of inflection points in the fitted time-current curve, and calculating the BOD with reference to Formula 1;
Formula 1; wherein
in Formula 1: Q₀ represents a reference electric quantity of the MEC reactor, in C; when the volume of the bioanode accounts for not less than 80% of that of the cavity of the reactor body, Q₀ is 3,000 C; when the volume of the bioanode accounts for 60% to 80% of that of the cavity of the reactor body, Q₀=150V+4200, and V represents a volume ratio, in %;
n represents the number of inflection points in the fitted time-current curve with a value of 0 to 2 according to an actual situation, and is dimensionless;
εᵢ represents a weighting coefficient for each of the characteristic declining stages in the fitted time-current curve, and is dimensionless; when n=0, i=1, ε₁=1; when n=1, i=1, 2, ε₁=0.4, ε₂-0.6; and when n=2, i=1, 2, 3, ε₁=0.2, ε₂=0.2, ε3=0.6;
F represents a Faraday's constant, 96,500 C/mol;
σ represents a total oxygen efficiency, in mol ·L/mg;
I_{F} represents a fitted curve, in mA;
tᵢ₋₁ represents a starting time point of an i-th characteristic decline curve, in s;
tᵢ represents an ending time point of the i-th characteristic decline curve, in s;
BOD represents a calculated BOD value, in mg/L; and
a calculation process of the total oxygen efficiency σ comprises: determining a biochemical oxygen demand after 5 days (BOD₅) value of the organic wastewater to be tested using a dilution and seeding method, and then substituting the BOD₅ value into Formula 1 to calculate the total oxygen efficiency σ.

10. The rapid test method according to claim 9, wherein a volume of the bioanode accounts for not less than 60% of that of the cavity of the reactor body.

11. The rapid test method according to claim 9, wherein the cathode is in close contact with an inner wall of the reactor body.

12. The rapid test method according to claim 10, wherein the cathode is in close contact with an inner wall of the reactor body.

13. The rapid test method according to claim 9, wherein the cathode is prepared from a titanium-based material; and the titanium-based material has titanium with a mass content of greater than 80%.

14. The rapid test method according to claim 9, wherein the cathode is in a mesh structure, and the mesh structure has a mesh number of not less than 100 mesh.

15. The rapid test method according to claim 13, wherein the cathode is in a mesh structure, and the mesh structure has a mesh number of not less than 100 mesh.

16. The rapid test method according to claim 9, wherein the bioaffinity material is a carbon-based material.

17. The rapid test method according to claim 9, wherein before the organic wastewater to be tested is introduced, the rapid test method further comprises: draining a liquid in the MEC reactor, and cleaning the cavity of the MEC reactor with deionized water.

18. The rapid test method according to claim 10, wherein before the organic wastewater to be tested is introduced, the rapid test method further comprises: draining a liquid in the MEC reactor, and cleaning the cavity of the MEC reactor with deionized water.

19. The rapid test method according to claim 9, wherein the nutrient solution has a pH value of 7.2; and the nutrient solution has the sodium acetate at a concentration of 0.1 mg/L to 0.5 mg/L.

20. The rapid test method according to claim 9, wherein the organic wastewater to be tested has a BOD₅ value of greater than 5 mg/L and does not have a biologically toxic substance.
